(19) United States
(12) Patent Application Publication
(10) Pub. No.: US 2010/0284951 A1
(43) Pub. Date: Nov. 11, 2010

(54) Novel compositions for the treatment of wounds and skin care

(76) Inventors: Thavisith Pongprapansiri, Bangkok (TH); Pensri Chatikavanit, Bangkok (TH); Somchai Boonchuen, Bangkok (TH)

Correspondence Address: Thavisith Pongprapansiri, 650 N. Rose Dr. #628, Placentia, CA, 92870, US

(21) Appl. No.: 12/436,796

(22) Filed: May 7, 2009

(51) Int. Cl. A61K 33/24; A61Q 19/00; A61K 33/34; A61K 33/32; A61K 33/26; A61K 33/14

(52) U.S. Cl. 424/62; 424/617; 424/630; 424/641; 424/646; 424/667; 424/680

(57) ABSTRACT

The present invention provides novel compositions which comprise sodium hypochlorite, sodium, chloride, both organic and inorganic trace minerals in hypertonic condition presented in the forms of a clear aqueous solution (low or high concentrations for further reconstitution with purified water to obtain the final ready-to-use solution), gel or salve, lotion, wet pad, wet foam, ointment, stick, and dry mixture solution, for local application for the purposes of cleansing, disinfecting and inhibiting infection and promoting the natural healing of conditions such as fresh cuts, surgical stitches, chronic (diabetic gangrene) and acute wounds, herpes zoster, athlete's foot, and bed sores, without the necessity for covering up the wounds with gauzes/plasters nor any concurrent use of topical medication. These compositions are also suitable for the treatment of conditions such as unpleasant body odor, acne and mixed bacterial infection in the ear.

### FIELD OF THE INVENTION

The present invention provides novel compositions for local application for the purposes of cleansing, disinfecting and inhibiting infection and promoting the natural healing of conditions such as fresh cuts, surgical stitches, chronic (diabetic gangrene) and acute wounds, herpes zoster, athletic foot, and bed sores, without the necessity for covering up the wounds with gauzes, plasters nor any concurrent use of topical medication.

### BACKGROUND OF THE INVENTION

It was commonly known that skin wound is generally characterized with a gap in the skin tissue and as the wound heals the gap in the wound is slowly and gradually filled in by new skin tissues which appear across the surface of the open wound so that when the healing process is complete, new skin tissues completely cover the former open area of the wound. Such process is medically referred to as a granulation process. However, these granulation tissues are somewhat fragile and rupture easily during the healing process as evidenced by the fact that when the conventional wound plasters or dry gauze dressings are removed from such skin lesions for cleansing and re-dressing, thus often interrupting and delaying the healing process. Most wounds, especially the infected and chronic wounds must be cleansed daily, depending on the degree of the wound condition and also depending on the amount of fluids in the wound. The more moist and softer the wound, the more often the wound has to be cleansed and re-dressed, while the replacement of dressing is a great danger of rupturing the fragile granulation tissues especially during the removal and replacement process, resulting in an unfavorable longer healing time and the risk of secondary infection. It was also commonly known that healing of chronic wounds can be problematic, especially in the case of diabetic mellitus and immune disorders. Chronic wounds affect an estimated four (4) million people a year in the U.S.A. resulting in health care costs in the billions of dollars. Acute and chronic wounds such as pressure ulcers (decubitus or bed sores) are the most common form of skin damage and occur when externally applied pressure exceeds capillary closing pressure. With the unrelieved pressure, tissue damage occurs, creating a wound. This type of wound is commonly found in patients who are confined to bed or wheelchairs. Prior to this novel invention, healing of such wound is perceived as almost impossible as long as the patients are confined to their bed and wheelchairs. In the U.S.A. alone, it is estimated that the treatment of pressure ulcers costs the U.S. healthcare system over 8.5 billion dollars annually (U.S. Pat. No. 5,958,420). Diabetic ulcers are sores that occur on the foot of a patient with diabetes. They have many contributing factors including trauma, poor circulation, and an abnormality of the nervous system. Current treatment protocols may involve the use of zinc oxide to accelerate healing and also the use of products that provide a moist wound environment. Prior to this novel invention, the healing time of such diabetic-related wound can be months and some times years, depending on the diabetic condition of the individuals. In serious cases, the condition of the wound could aggravate so bad that the wound could become deeper and spreads and never dries up, ending up with an extremely unpleasant odor in the wound. Such conditions may lead to amputation of the toes or feet, and if the wound still spreads further, amputation at knee and thigh height may be recommended in order to prevent further spread of the wound.

The theory of keeping a moist wound environment in the treatment was reported in 1971, as a result of experiments which showed that by keeping the wound surface damp, healing was accelerated. The first hydrogel was, therefore, introduced in 1985. Many now consider a moist environment preferable in the treatment of wounds, because research data suggest that the body's natural healing processes of autolytic debridement, re-epithelialization, granulation of the tissues, and remodeling/maturation are enhanced in a moist environment. According to the report, healing can occur more quickly in a moist healing environment than in wounds left exposed to air. However, the threat of infection remains an ongoing concern, and typically, products that are bacteriostatic unfortunately are also cytotoxic. In other words, they inhibit or stop the growth of new tissue cells, thus slowing the wound-healing process. Therefore, many health care providers do not use a moist protocol for infected wounds because a moist environment has been shown to be conducive to growth of bacteria and causes unpleasant odor and has become a concern of health care providers. It would, therefore be advantageous to provide methods and apparatus for improved moist treatment of an infected acute or chronic wound that would inhibit the growth of bacteria, while not inhibiting the growth of new tissue cells. Commercial wound cleansers consist principally of two parts: an active agent and a carrier. The active ingredients generally possess surface active effects and are known to be toxic. For example, Dakin's solution, a buffered 0.5 percent solution of sodium or potassium hypochlorite, which was introduced since World War I as an antiseptic irrigant for wound, is later known to be injurious to tissues, and is therefore considered more a debriding agent than a cleansing one. Its antimicrobial action is due to release a hypocholorous acid, which is active in neutral or acidic solutions but is less active in alkaline solutions.

### SUMMARY OF THE INVENTION

Prior to this invention, very commonly available treatments for fresh cuts, surgical stitches, infected and chronic (diabetic-related) wounds, bed-sores, herpes zosters, athletic foots and infection of the ears involves daily cleansing of the wounds/symptoms with conventional alcohol or hydrogen peroxide (which has little value as an antiseptic and is more effective as a sporicide than a bactericide and even at low concentration is damaging to tissues) coupled with an iodine-based antiseptic application (which is known to produce a strong astringent effect) and mostly to be followed by a topic application of an antibiotic ointment. Most iodine-based antiseptic is known to be a strong oxidizing agent, therefore, the cell membrane of the tissue around the wound area may also be irritated and can be injured along with the cell membrane of the bacteria, which could possibly result in the inflammation of the tissue in the wound area, thus causing a delayed wound healing process. In addition, oral medication is simultaneously prescribed along with the daily wound cleansing as described in the case of infected wounds. Applicants have now discovered novel formulations which are bioacceptable and safe even to the softest tissues, which make it possible for all the aforementioned wounds and skin infections/conditions, some of which were regarded, prior to this invention, as almost impossible to heal naturally and rapidly without the need to apply topical medication and without the need of covering up the wound with gauzes/plasters. Apart from that, wounds using these novel formulations as treatment may be in contact with water on a daily basis without any fear of infection. These formulations could be made available in the forms of a clear aqueous solution (low or high concentrations for further reconstitution with purified water to obtain the final ready-to-use solution), gel or salve, lotion, wet pad, wet foam, ointment, stick, dry mixture with solution; for local application for the purposes of cleansing, disinfecting and inhibiting infection and promoting the natural healing of conditions such as fresh cuts, surgical stitches, chronic (diabetic gangrene) and acute wounds, herpes zoster, athletes foot, and bed sores, without the necessity for covering up the wounds with gauzes/plasters nor any concurrent use of topical medication. These compositions are also suitable for the treatment of conditions such as acne and mixed bacterial infection in the ear. This rapid and natural healing process includes: a rapid autolytic debridement re-epithilialization, granulation and maturation of the tissue cells in the wound area and it also creates a novel apparatus for treating wounds with a clean environment while inhibiting microbial infection and avoiding the unpleasant odor in diabetic related or chronic wounds.

### EXAMPLES OF STUDIES

### Example 1

Healing time for fresh cuts: Prior to this invention, healing time is usually 2-3 days for a small fresh cut or longer if the cut is larger and requires surgical stitching, or when infection occurs during the healing process. However, in the case of infected wounds, healing time of the cut may take up to 5-6 days. Observational studies on the healing time of fresh cuts showed these cuts would naturally heal within 2-3 days after topical application by dampening the wound area 2-3 times daily with a clear aqueous solution described in this invention, without any concurrent use of any other topical medication.

### Example 2

Healing time for surgical stitches: Prior to this invention, healing time is usually 4-5 days or longer depending on the size and whether or not the stitches have become infected. Daily cleansing with the conventional alcohol/hydrogen peroxide and topical ointment application coupled with 4-5 days of oral antibiotic medication is usually recommended. Observational studies showed these stitches would naturally heal within 2-3 days after topical application with a clear aqueous solution described in this invention by dampening the wound area, 1-2 times daily, without any concurrent use of any other topical medication.

### Example 3

Healing time for infected wounds: Prior to this invention, whenever a wound becomes infected, complication occurs in such a way that the wound has to be cleansed daily with alcohol and hydrogen peroxide, and topical ointment application coupled with 4-5 days of oral doses of antibiotic, or until the wound completely granulates and is healed. The healing time of such wound can take a week or longer especially when bleeding and pus are present in the wound. Observational studies showed these infected wounds would naturally heal within 3-4 days after topical application with a clear aqueous solution described in this invention by dampening the wound area, 2-3 times per day, without any concurrent use of any other topical medication.

### Example 4

Healing time for chronic (diabetic related) wounds: Prior to this invention the healing time of such wound can be months and sometimes years, depending on the diabetic condition of the individuals. In serious cases, the condition of the wound could aggravate so bad that it will spread from the toes area to the ankle area and produces extremely unpleasant odor and never dries up and such conditions may lead to amputation of the area above the wound area, such as amputation of the toes or feet or as high as the knee and thigh. To assure an aseptic condition of diabetic gangrenes and to avoid air/contact infection, health care providers usually recommend the wound to be covered-up with gauzes. Observational studies showed this chronic (diabetic-related) wound would rapidly and naturally dry up within 4-6 weeks right after topical application with a clear aqueous solution described in this invention by thoroughly dampening the wound area 2-3 times per day. At the same time the unpleasant odor from the wound area would also rapidly disappear.

### Example 5

Healing time for herpes zoster (viral infection): Prior to this invention, in freshly viral-infected herpes zoster cases, an anti-viral topical ointment application is usually recommended along with medical prescription for a period of 5 days. Healing time is usually a week or longer. But observational studies showed a natural healing of herpes zoster within 3-4 days after topical application with a clear aqueous solution described in this invention, 2-3 times per day, without any concurrent use of any other topical medication. In more severe viral-infected cases of herpes zoster, it is very common that only anti-viral topical ointment application is recommended without oral medical prescription. The wound is usually left to heal naturally but the healing process can take weeks or months or it can also worsen and become very painful for the infected individuals. Observational studies have also shown a rapid and natural healing of these severe herpes zoster cases within 3-4 days after topical application with a clear aqueous solution described in this invention, twice to three times per day, without any concurrent use of any other topical medication.

### Example 6

Healing time for bedsore: Prior to this invention, bedsore is known to be a chronic problem and is almost impossible to cure. Observational studies showed a natural healing of bed sore within 3-5 days after topical application with a clear aqueous solution described in this invention, 2-3 times daily, without any concurrent use of any other topical medication.

### Example 7

Healing time for athletic foot; Prior to this invention, athletes foot is usually treated with antifungal powder or ointment and it usually takes a couple of days for the infected area to heal. Observational studies showed a natural healing of athletes foot within 2-3 days after topical application with a clear aqueous solution described in this invention, 2-3 times daily, without any concurrent use of any other topical medication.

### Example 8

Healing time for “vulgaris” acne: Prior to this invention, treatment of acne by antibacterial solutions, lotions or gels twice a day is very common, healing time is between 1-2 months. In more severe cases, oral medication is commonly used in acne treatment along with topical medication. Bacteria resistance often occurs, therefore, switching of antibiotic may be necessary. In such cases, healing time can take up to 3-4 months. Observational studies showed, in nearly all the test cases, a rapid and natural healing (within 3-5 days) of “vulgaris” acne after a few drops by topical application with a clear aqueous solution described in this invention twice daily. These healings were observed with and without any concurrent use of topical medication.

### Example 9

Healing time for ear infection: Prior to this invention, treatment of ear infection is usually with daily cleansing of the debris by suction followed by ear drops containing one of these medications such as antibiotic, antifungal or antiviral drugs and it usually takes weeks for the ear to dry up and heal. Observational studies showed, in nearly all the test cases, a rapid and natural healing (within 3-5 days) of mixed-infection ears after a few drops by topical application with a clear aqueous solution described in this invention, 2-3 times daily. These healings were observed with and without any concurrent use of topical medication.

### EXAMPLES OF COMPOSITIONS IN THE CLEAR AQUEOUS SOLUTION

### Example 1

Sodium hypochlorites 0.25 g.

Sodium Chloride 1.50 g.

Copper Sulfate 10.0 mg

Zinc Sulfate 15.0 mg

Magnesium Sulfate 20.0 mg

Purified Water q.s. to 1000 cc

### Example 2

Sodium hypochlorites 0.5 g.

Sodium Chloride 2.5 g.

Copper Sulfate 25.0 mg

Zinc Sulfate 35.0 mg

Magnesium Sulfate 50.0 mg

Purified Water q.s. to 1000 cc

### Example 3

Sodium hypochlorites 1.25 g

Sodium Chloride 300 g

Copper Sulfate 50.0 mg

Zinc Sulfate 200.0 mg

Magnesium Sulfate 100.0 mg

Purified Water q.s. to 1000 cc

### DETAILED DESCRIPTION OF THE INVENTION

The present invention provides novel osmotically active compositions which comprise: 1) a mild and low concentration of sodium hypochlorite for its mild bleaching, antimicrobial activity and to cleanse the debris and dead tissues without injuring the tissue cells and to inhibit the autolytic enzyme activity, 2) a hypertonic condition formulation for its bacteriostatic and bacteriocidal activities for the control of bacteria infection with a balanced cation (+) sodium and anion (−) chloride which also possesses bacteriostatic and bacteriocidal properties, and, 3) sulfur from sulfate salt of sodium, potassium, magnesium, zinc, iron, copper, manganese to stimulate the enzymatic process and to promote the rapid healing activity of white blood cells resulting in a rapid fibroblast, granulocyte, monocyte infiltration in and around the wound area thus stimulating a rapid protein matrix and re-epithelialization of the tissue cells. One characteristic of this novel skin healthcare composition is that it comprises a mild and low concentration of sodium hypochlorite at 0.025-0.125% by weight when compared with the total weight of the composition, sodium chloride 1-30% by weight when compared with the total weight of the composition, sulfur from sulfate salt of minerals such as at 10-350 ppm. The terminology “a novel skin health-care composition” mentioned herein and in the Claims of this patent is intended to mean composition comprising all the aforementioned ingredients which have been proven to show favorable efficacy in preventing and treating skin infections caused by bacteria, fungus, ear infection, acne and for an effective control of unpleasant body odor as well as its efficacy to promote a rapid fibroblast, granulocyte, monocyte infiltration in and around the wound area which helps to stimulate a rapid protein matrix and re-epithelialization of the tissue cells.

These novel compositions may be in clear aqueous solution, or a concentrated aqueous solution that can be constituted with water to obtain the final aqueous solution, gel or salve, lotion, wet pad, wet foam, ointment, stick, dry mixture with solution and are formulated for all-purposes local application, i.e., cleansing, inhibiting infection and promoting the natural healing of conditions such as fresh cuts, surgical stitches, chronic (diabetic gangrene) and acute wounds, herpes zoster, itching in athlete's foot due to fungus and bed sores. These compositions are also suitable for the treatment of conditions such as unpleasant body odor, acne and bacterial infection in the ear. These novel formulations make it possible for all the aforementioned wounds and skin infections/conditions which were very difficult or almost impossible to heal to rapidly and naturally heal with and without the need for any concurrent use of topical medication and without the need of covering up the wound with plasters or gauzes. This rapid and natural healing process includes: an autolytic debridement re-epithilialization, granulation and maturation of the tissue cells in the wound area and it also creates a novel apparatus for treating wounds with a clean environment while inhibiting microbial infection while avoiding unpleasant odors. Prior to this invention, very commonly available treatments for fresh cuts, infected and chronic wounds involves daily cleansing of the wounds with conventional alcohol or hydrogen peroxide (which has little value as an antiseptic and is more effective as a sporicide than a bactericide and even at low concentration is damaging to tissues) coupled with an iodine-based antiseptic application (which is known to produce a strong astringent effect) and mostly to be followed by a topic application of an antibiotic ointment. Most iodine-based antiseptic is known to be a strong oxidizing agent, therefore, the cells membrane of the tissue around the wound area may also be irritated and can be damaged along with the cell membrane of the bacteria, which could possibly result in the inflammation of the tissue in the wound area, thus causing a delayed wound healing process. In addition, oral medication is usually prescribed along with the daily wound cleansing as described in the case of infected wounds.

This invention has created compositions which are bio-acceptable and safe to the softest tissues which may even be used as eye-drops, therefore, provide assurance of no side-effects when used for a prolonged period of time. Listed hereunder are some examples of the compositions as per the claims in this patent and outlines of the benefits. However, it should not be construed to mean that the patent protection is only limited to the compositions as shown in the examples demonstrated herein.

What is claimed is:

1. A method of preparing a novel skin health-care composition comprised of mild and low concentration of sodium hypochlorite at 0.025-0.125% by weight when compared with the total weight of the composition, sodium chloride 1-30% by weight when compared with the total weight of the composition, sulfur from sulfate salt of minerals such as at 10-350 ppm.

2. The method according to claim 1 to formulate compositions which possess both organic and inorganic minerals such as sodium, magnesium, potassium, zinc, iron, iodine, zinc, copper, selenium, sulfur or sulfate salt.

3. The method according to claim 2 to formulate compositions with antiseptic and antimycotic, dermatic and or light absorbent substances.

4. The method according to claim 3 to formulate compositions with herbal roots, herbal leaves extracts and/or essential oil.

5. The method according to claim 4 to formulate compositions with pH adjuster arid/or pH stabilizer.

6. The method according to claim 5 to formulate compositions with any of these additives such as water, preservative, detergent, surfactant, zinc oxide, skin protectant, whitening, bleaching, oxidizing, reducing, soothing, sudsing and buffering agents.

7. The method according to claim 6 to formulate compositions with osmolarity regulator, glycerine, osmotic adjuster, tissue granulation stimulator and/or, astringent inducing and/or cells proliferation stimulator.

8. The method according to claim 7 to formulate compositions with any of these substances such as anti-inflammation, anti-viral, anti-biotic, antioxidant, organic acid, bio-fermented products from micro organisms, fulvic acid, humic acid, anti-fungal and/or antimicrobial drugs.

9. The method according to claim 8 to formulate compositions with any of these substances such as amino acids, vitamins and minerals.

10. The method according to claim 9 to formulate compositions with beta glucan or with keratine Derivatives.

11. The method according to claim 10 to formulate compositions with any of these aqueous solutions, high concentration of aqueous solution further constituted with water to obtain the final aqueous solution, greaseless soluble or salve gel-water, lotion, wet pad, cream-based preparation, petrolatum-based ointment, emulsion-based and/or foam-based preparation.

12. The method according to claim 10 to formulate compositions with moistened towelett, stick and/or dry mix for further reconstitution in water prior to application.

13. The method according to claim 10 to formulate compositions on plasters or gauzes.

14. The method according to claim 10 to formulate compositions for use as wound cleansing solution in fresh cuts, infected wounds, surgical stitches, chronic diabetic gangrene, bed sores, herpes zoster, itching in athlete's foot due to fungus, eye drops and ear drops due to infection, nasal drop and nasal spray, eye cleansing solution, rectal/vagina cleansing, and for the control of unpleasant body odor and skincare.
